(19) United States
(12) Patent Application Publication
(10) Pub. No.: US 2021/0219565 A1
(43) Pub. Date: Jul. 22, 2021

(54) METHODS OF MAKING COFFEE WITH HIGH AMOUNTS OF PHENOLS AND CHLOROGENIC ACIDS

(71) Applicant: Robert Burns Arnot, Stowe, VT (US)

(72) Inventor: Robert Burns Arnot, Stowe, VT (US)

(21) Appl. No.: 17/185,348

(22) Filed: Feb. 25, 2021

Related U.S. Application Data
(63) Continuation-in-part of application No. 15/275,589, filed on Sep. 26, 2016, now abandoned.
(60) Provisional application No. 62/222,821, filed on Sep. 24, 2015.

(51) Int. Cl. A23F 5/04; A23F 5/08; G01N 33/00; G01N 30/72

(52) U.S. Cl. CPC A23F 5/04; A23F 5/08; A23F 2200/00; G01N 30/72; G01N 33/0098

(57) ABSTRACT

A method of making coffee with high amounts of phenols and chlorogenic acids is provided. Coffee beans are tested for phenols and chlorogenic acids and selected if they are found to have predetermined levels of phenols and chlorogenic acids. The selected coffee beans may be treated and then shipped where they are roasted at a temperature and for a time period that preserves the phenols and chlorogenic acids therein without adversely affecting the taste and aroma thereof. The roasted coffee beans are then ground to a selected particle size and are brewed with water at a temperature and for a time period to produce liquid coffee having high amounts of phenols and chlorogenic acids that also has favorable flavor.

### FIELD OF THE INVENTION

The present invention generally relates to methods of making coffee having beneficial amounts of phenols, chlorogenic acids (CGAs), antioxidants and anti-inflammatory compounds.

### BACKGROUND

As shown in FIG. 1, coffee delivers the highest antioxidant content in the American diet, but the vast majority of coffee delivers far fewer antioxidants and anti-inflammatory compounds than it could and as such delivers very little benefit with a host of negatives from high caffeine levels and fats to Acrylamides and HHQ. When tested for improvements in core health, most coffees have either a negative affect or a neutral affect, meaning that they are either of no benefit to the consumer or pose a cardiovascular risk over time. This has been confirmed by tests that show most coffees have a neutral or negative affect on endothelial cell function and vascular function when tested in humans.

In a 2015 Advisory Report to the Secretary of Health and Human Services and the Secretary of Agriculture, the Dietary Guidelines Advisory Committee of the USDA stated as follows: “Consistent evidence indicates that coffee consumption is associated with reduced risk of type 2 diabetes and cardiovascular disease in adults. Moreover, moderate evidence shows a protective association between caffeine intake and risk of Parkinson's disease. Therefore, moderate coffee consumption can be incorporated into a healthy dietary pattern, along with other healthful behaviors.” An influential panel of experts convened by the World Health Organization concluded in June, 2016 that regularly drinking coffee would protect against at least two types of cancer and also concluded that there was a lack of evidence that it might cause other types of cancer.

Significant research has confirmed that habitual flavonoid intake can improve heart health, improve blood sugar clearance, decrease fat absorption, and maintain weight loss, as well as other benefits as can be seen in FIGS. 2-3.

Accordingly, a need exists for methods of preparing coffee that is robustly healthy for most people and that they will still enjoy with great taste and aroma. The methods of the present invention meet this need.

### SUMMARY OF THE DISCLOSURE

A method of preparing a liquid coffee drink includes determining amounts of polyphenols in a plurality of coffee beans using mass spectrometry, selecting coffee beans from the plurality of coffee beans that contain between 20,000 mg/kg and 40,000 mg/kg of 3 caffeoylquinic acid (3 CQA), roasting the selected coffee beans for a time period of 7.5 to 12 minutes at a temperature of 390° F. to 412° F., wherein the roasted coffee beans have between 11,000 and 22,000 mg/kg of 3 CQA. The roasted coffee beans are then ground to a particle size of between 100 microns and 1200 microns and the roasted ground coffee is brewed in water at 210° F. for about 3.5 minutes to produce the liquid coffee drink, wherein the liquid coffee drink includes at least 258 mg of 3 chlorogenic acid per 250 mL of the liquid coffee drink, at least 141 mg of 4 chlorogenic acid per 250 mL of the liquid coffee drink, and at least 99 mg of 5 chlorogenic acid per 250 mL of the liquid coffee drink.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a chart showing the top ten sources of antioxidants in the U.S. diet;

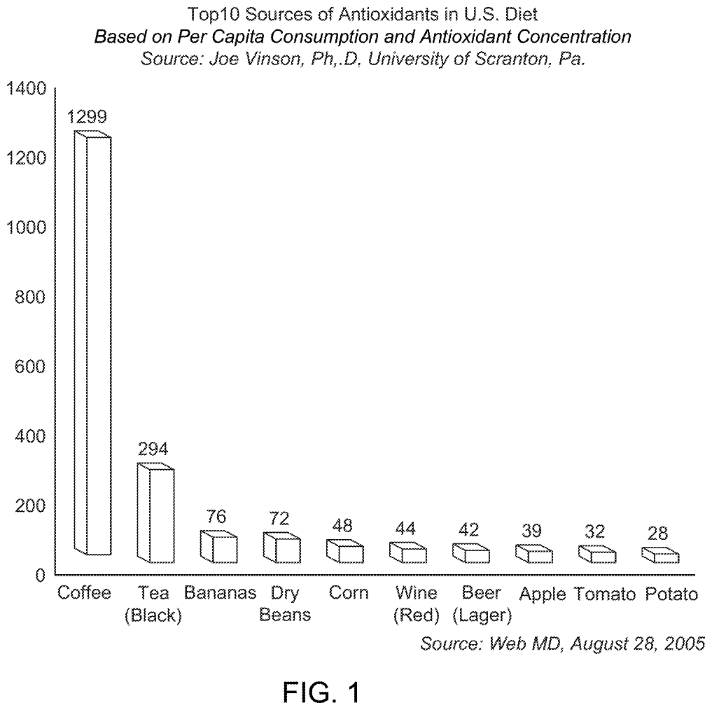

FIG. 2 is a chart showing that caffeine consumption by women can lessen depression;

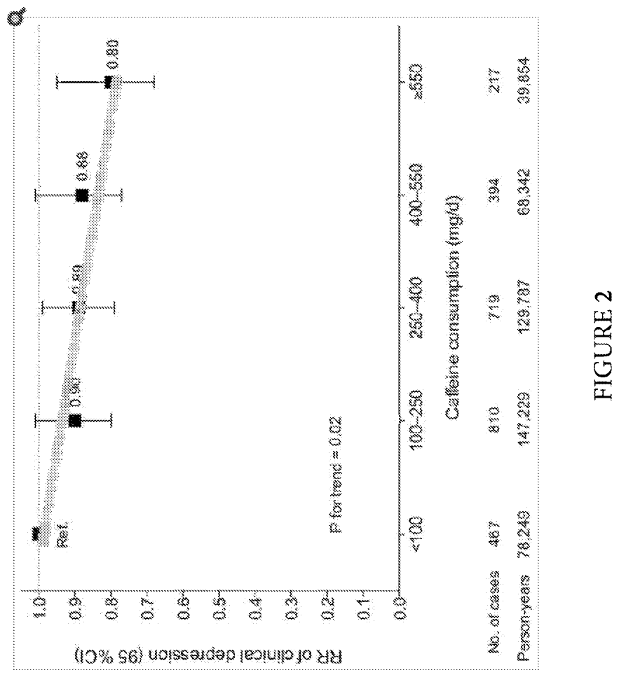

FIG. 3 is a chart showing that phenol intake can result in a 30% decrease in all causes of death; and

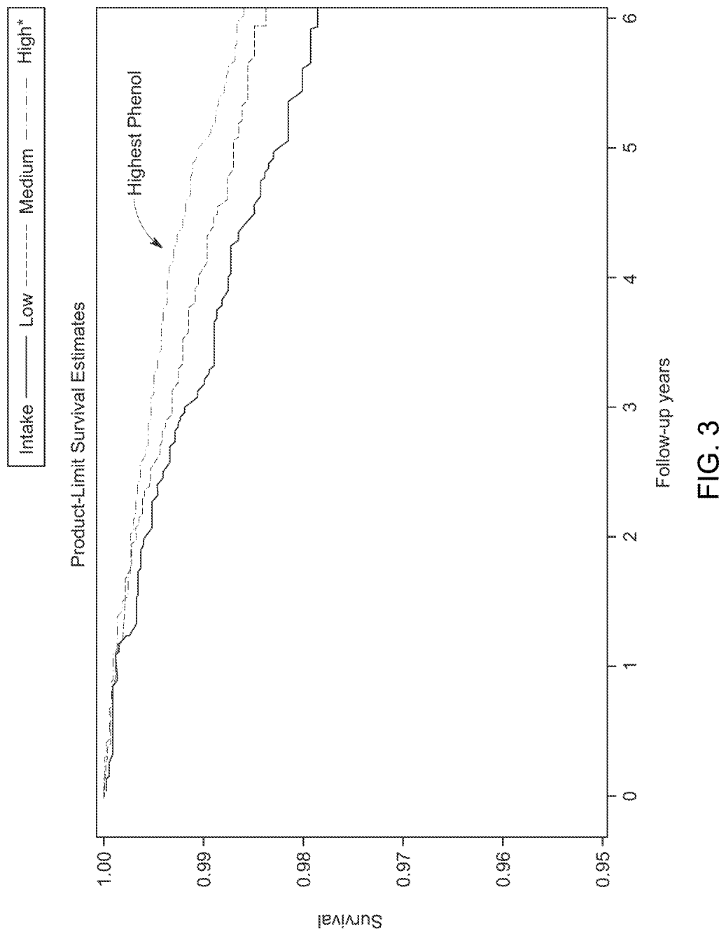

FIG. 4 is a chart showing that certain roast temperatures can decrease acrylamide levels.

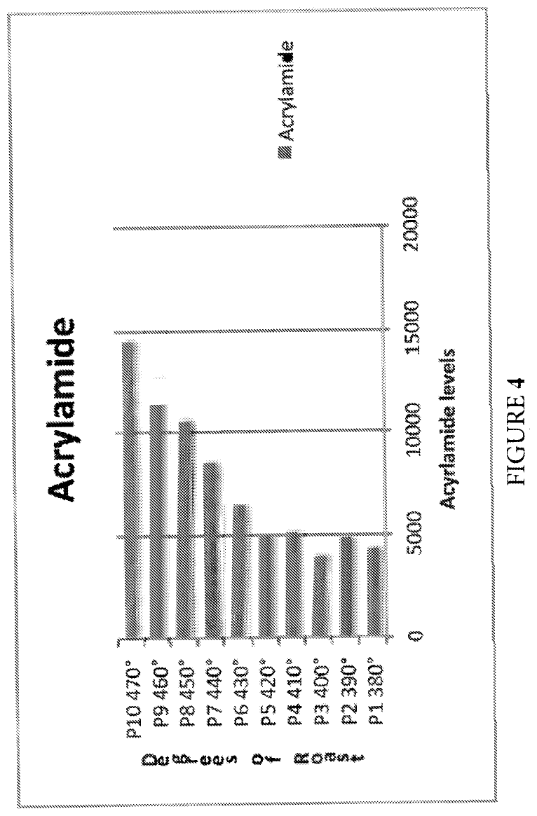

### DESCRIPTION OF THE DISCLOSURE

Coffee having high amounts of phenols and chlorogenic acids is prepared by obtaining coffee beans having a high amount of phenols, CGAs, and antioxidants. The amounts of phenols, CGAs, and antioxidants may be determined by any suitable analytical technique, such as High Performance Liquid Chromatography or Gas Chromatograph Mass Spectrometry. Coffee beans determined to have desired amounts of phenols, CGAs, and antioxidants are then prepared and shipped in a manner that adequately maintains their nutritional profiles. For example, the selected beans may be washed and vacuum packed prior to shipment. Once delivered to a roasting facility, these coffee beans are roasted for an appropriate roast time and temperature that preserves the amount of phenols, CGAs, and antioxidants at higher levels while not negatively impacting the aroma of the beans or the flavor of coffee made from them. The roasted beans are then ground to a size that presents sufficient total surface area of the ground beans such that, when exposed to water during brewing, CGAs are adequately extracted and a good taste profile results in the coffee. Brewing techniques are then employed that result in sufficient extraction of CGAs, e.g., over 350 mg of chlorogenic acid (3, 4 and 5 caffeoylquinic acid) per cup of coffee.

### 1. Testing and Selection of Coffee Beans Having a High Amount of Phenols, CGAs and Antioxidants

Various regions in Africa, Indonesia and South America were identified by looking at the right mix of altitude, latitude, soil, terroirs, and cloud cover for coffee beans that would produce the highest natural polyphenol levels. It was determined that beans which are stressed the most protect themselves by manufacturing the largest amount of polyphenols. Accordingly, coffee beans were obtained from various regions in Brazil, Kenya, Ethiopia, Columbia, Rwanda, Congo, and Indonesia where coffee was grown at high altitude. Table 1 shows green beans tested by HPLC with significant variation in polyphenol levels from a high of 41,844 to a low of 13,595 mg/kg.

TABLE 1

| | | Chlorogenic Acid |
|---|---|---|
| Sample | Roast | mg/Kg (3 CGA) |
| 1 | Green Coffee Beans | 41844 |
| 2 | Green Coffee Beans | 39147 |
| 3 | Green Coffee Beans | 38632 |
| 4 | Green Coffee Beans | 36036 |
| 5 | Green Coffee Beans | 35962 |
| 6 | Green Coffee Beans | 35896 |
| 7 | Green Coffee Beans | 33342 |
| 8 | Green Coffee Beans | 29434 |
| 9 | Green Coffee Beans | 28641 |
| 10 | Green Coffee Beans | 27010 |
| 11 | Green Coffee Beans | 26906 |
| 12 | Green Coffee Beans | 26676 |
| 13 | Green Coffee Beans | 26023 |
| 14 | Green Coffee Beans | 24737 |
| 15 | Green Coffee Beans | 24092 |
| 16 | Green Coffee Beans | 23869 |
| 17 | Green Coffee Beans | 23533 |
| 18 | Green Coffee Beans | 22623 |
| 19 | Green Coffee Beans | 21908 |
| 20 | Green Coffee Beans | 20900 |
| 21 | Green Coffee Beans | 20816 |
| 22 | Green Coffee Beans | 20200 |
| 23 | Green Coffee Beans | 18417 |
| 24 | Green Coffee Beans | 18368 |
| 25 | Green Coffee Beans | 17916 |
| 26 | Green Coffee Beans | 15996 |
| 27 | Green Coffee Beans | 14643 |
| 28 | Green Coffee Beans | 13993 |

Also, the soil proved to be important so that certain beans had nearly double the phenol content of other beans in the same area. It was determined that volcanic soil also produces a high mineral and nutritional base for growing coffee.

2. The Preparation and Shipment of Selected Coffee Beans to Maintain their Polyphenol Levels

Once coffee beans are selected and picked, their preparation plays an important role in how well they can maintain their nutritional profiles in shipping. Since the selected coffee beans may have a long trip by ship, for example, where they are exposed to heat, humidity, and water, they are vacuum packed or placed in protective packing or the like to maintain their nutritional profiles in shipping.

### 3. The Roasting of the Selected Coffee Beans

It was determined that the roasting of the selected coffee beans for a time period of 7.5 to 12 minutes and a temperature range of 390° F. to 412° F., preserved the highest amount of phenols, CGAs, and antioxidants. The following Table 2 identifies the top selected coffee beans for total CGAs and phenols after roasting. This is significant because it shows the preservation of CGAs by the roast technology of the present invention. It is noted that the Ethiopian Homacho bean had the highest CGA after roasting. All commercially available coffees failed to deliver adequate amounts of CGAs and phenols after roasting, as shown in Table 2, with many popular brands bought in a grocery store or the like and tested in an independent laboratory by HPLC.

TABLE 2

| | Chlorogenic Acid |
|---|---|
| | mg/Kg |
| Roasted Coffee | 3 Caffeoyliquinic acid |
| Ethiopian Homacho | 24408 |
| Ethiopian Hambella | 22817 |
| Kenya Karagoto | 22291 |
| Nyerl Chainara | 21425 |
| Kenya Karagoto | 20766 |
| Gibsons 8B | 20567 |
| Dr. Danger Hard Core (Extra Light) | 18198 |
| P15 Ethiopia Adado | 18109 |
| Decaf Ethiopia Sidama Natural | 13256 |
| Kenya Murage | 15997 |
| P11 Mexico Chipas | 15338 |
| P12 Brazil Sitio Bella Vista | 14864 |
| Daktari Coffee Diamond Glacier | 13013 |
| Kenya aa tambaya gran pro | 12956 |
| P19 Night OPS | 12128 |
| Colombian Hulla Excelso | 12118 |
| P13 Rwanda Kabirizi | 12019 |
| Dr. Danger Surgical Strike | 11984 |
| Brio Rwanda Kabirizi Washed Beans | 11721 |
| Kenya aa + grain pro | 11658 |
| Kenya Karagoto | 11600 |
| P17 Indian Cherry Robusta | 11586 |
| Blue Isle Columbia Hulla | 11410 |
| Kenya nyerl wachurl | 10933 |
| Gevalia Traditional mild | 11900 |
| Dunkin Donuts Original Blend K Cup | 11656 |
| Dunkin Donuts Original Blend | 11042 |
| Lavazza Kilimanjaro | 8777 |
| P16 Bulletproof | 8699 |
| McCafe Decaf Medium Roast | 8668 |
| Counter Culture | 8577 |
| Rockin & Roastin Medium Roast | 8248 |
| Starbucks Veranda Blend Blonde K Cup | 8135 |
| Green Mountain Coffee Breakfast Blend K Cup | 8133 |
| Eight O'clock Coffee Colombian Medium | 8027 |
| Melltta Classique Supreme Medium | 7247 |
| Folgers Classic Roast | 7124 |
| Bullet Proof | 7119 |
| Newman's Own Breakfast Blend | 6885 |
| Green Mountain Coffee Breakfast Blend | 6603 |
| Kauai Coffee Koloa Estate Medium | 6592 |
| Green Mountain Coffee Southern Pecan | 6447 |
| Maxwell House Original Roast | 6227 |
| Speeder earls cifee speeder's blend | 5534 |
| Koffee kuit | 5447 |
| Peets Coffee House Blend Deep Roast | 2755 |
| Peet's Major Dickason's Blend Dark | 2750 |
| Kicking hourse Dark Roast | 2654 |
| Signature Select Costa Rica Medium | 2277 |
| Seattle's Best Coffee Signature Blend #3 | 2103 |
| Gevalia Kaffe French Roast | 2039 |
| Starbucks Caffe Verona Dark | 2026 |
| Starbucks Verona Dark K Cup | 1968 |
| Death Wish | 1907 |

The roast profile in Chart 1 below shows the destruction of polyphenols with increasing roast temperatures.

### 4. The Grinding of the Beans

It was determined that the grinding of the beans to maximize the total surface area exposed to water during brewing would provide maximal CGA extraction and a good taste profile. The extraction of CGAs from very coarse to powdered grinds was analyzed as well as measuring CGAs for grind size matched to brewing methods. It was determined that the finer the grind, the better the extraction of CGAs. The optimal grind sizes were found to be 100 microns to 1200 microns.

### 5. The Use and Testing of Brewing Techniques

Various brewing techniques were tested to determine those that provide the maximum extraction of CGAs, e.g., over 300 mg of 3, 4, and 5 chlorogenic acids to a cup of coffee (approx. 7 oz). The maximum in the published literature is 188 mg/cup. The results of testing brewing techniques at approximately 210° F. for about 3.5 minutes are illustrated in the following tables, showing in one instance values of 3, 4 and 5 CQA combined of over 1000 mg/cup.

The tables below (Tables 3A-3C) demonstrate the ability to extract the highest amounts of coffee polyphenols using commonly employed brewing techniques. These tables demonstrate that not only do the coffee beans that are tested, selected, roasted, and brewed in accordance with the present invention have the highest polyphenol levels reported, but that they also ultimately deliver these polyphenols into an actual cup of coffee that a consumer drinks. Table 3A reports the results in milligrams per kilogram of coffee used for the extraction. Table 3B reports the actual mg of polyphenols found in the brewed coffee itself in mg per liter of liquid coffee. Table 3C shows the actual extraction method used across a variety of brewing techniques and platforms and the resultant TDS score which best reflects how successful the extraction was according to the Specialty Coffee Association of America. In summary, the highest chlorogenic values ever delivered into a cup of coffee are shown for coffee that still meets the standard for superior taste.

In table 3A, the amount of chlorogenic acid extracted from coffee using a standard chemical extraction and processed using a high performance liquid chromatography device and is expressed in milligrams of these per kilogram of coffee. This shows that the finer the grind size is, the higher the extraction amount of total phenols.

TABLE 3A

| | | | | Total | Total |
|---|---|---|---|---|---|
| | | Grams | | Phenols | CGA and |
| | Grind | of | Chlorogenic | Non CGA | Non CGA |
| Brewing Method | Size | Coffee | Acid mg/Kg | mg/Kg | Phenols |
| 60B 1.0 Grind | 1 | 5 | 21543 | 37212 | 58755 |
| 5 g Turkish | | | | | |
| MR. COFFEE | 9 | 30 | 17021 | 29858 | 46879 |
| 60B 8.5 | | | | | |
| Grind 30 g | | | | | |
| KALITA 60B | 9 | 20 | 16696 | 29370 | 46066 |
| 8.5 Grind 20 g | | | | | |
| CHEMEX 60B | 10 | 30 | 16486 | 28451 | 44937 |
| 9.5 Grind 30 g | | | | | |
| KALITA 60B | 10 | 20 | 16617 | 27814 | 44431 |
| 9.5 Grind 20 g | | | | | |
| AEROPRESS | 5 | 14 | 15172 | 26680 | 41852 |
| 60B 5.0 | | | | | |
| Grind 14 g | | | | | |
| AEROPRESS | 7 | 14 | 14357 | 24586 | 38943 |
| 60B 7.0 | | | | | |
| Grind 14 g | | | | | |
| AEROPRESS | 7 | 22 | 13865 | 24158 | 38023 |
| 60B 7.0 | | | | | |
| Grind 22 g | | | | | |
| FRENCH | 11 | 25 | 12140 | 20664 | 32804 |
| PRESS 60B 10.5 | | | | | |
| Grind 25 g | | | | | |

The brewing methods listed in Table 3A are as follows:

60B Turkish: This uses a very fine grind put directly into boiling water as both Turkish and Greek coffees use for the highest possible extraction.

MR Coffee is a commonly available and inexpensive brewer.

Kalita is a cup and filter technique commonly used in homes and high end coffee shops.

Chemex is a glass funnel and filtration technique used in the white house during the Obama years which yields a very pleasant tasking cup of coffee.

Aeropress is a technique using a piston and cylinder requiring a lesser amount of coffee so that it is more cost effective.

French press is a popular technique using a coarser grind, which is reflected in lower CGA values.

Grind Size: These can vary from 1.0, which is a very fine espresso grind, to 11.0, which is coarse. These were determined in a reference laboratory

Grams of Coffee: Each technique specifies the grams of coffee necessary to meet the Specialty Coffee Association of America's correct coffee extraction for the most pleasant taste.

Total amount of chlorogenic acids, which is the main polyphenol found in coffee and thought to be responsible for its health effects.

The amount of non chlorogenic acid polyphenols is shown, which are also important to health.

The last column in Table 3A is the sum total of the chlorogenic acid polyphenols and the non chlorogenic acid polyphenols.

Table 3B shows the amount of chlorogenic acids and total polyphenols extracted using commonly available brewing techniques and therefore available to the consumer for consumption. All of the columns and rows are the same as those of Table 3A except for the addition off a column for total chlorogenic acid per 250 ml cup of coffee These values are the highest ever reported and therefore deliver the highest amount of polyphenols per cup of liquid coffee (as used herein, 250 ml). Prior amounts reported were a maximum of 188 mg of chlorogenic acids out of 104 different coffees, for example. As can be seen in Table 3B, a maximum of 462.25 per cup was found, with all brewing methods using the coffee beans tested, selected, and roasted in accordance with the present invention had at least 248 mg of total chlorogenic acid per 250 ml of liquid coffee drink, as determined with a high performance liquid chromatography device using standards purchased from a reference laboratory.

TABLE 3B

| | | | | Total | | Total |
|---|---|---|---|---|---|---|
| | | Grams | | Phenols | Total CGA and | Chlorogenic |
| Brewing | Grind | of | Chlorogenic | Non CGA | Non CGA | Acid Per |
| Method | Size | Coffee | Acid mg/L | mg/L | Phenols/Liter | 250 ml Cup |
| AEROPRESS | 1 | 5 | 1849 | 3221 | 5070 | 462.25 |
| 60B 7.0 | | | | | | |
| Grind 22 g | | | | | | |
| KALITA 60B | 9 | 30 | 1237 | 2176 | 3413 | 309.25 |
| 8.5 Grind | | | | | | |
| KALITA 60B | 9 | 20 | 1224 | 2152 | 3376 | 306.00 |
| 9.5 Grind | | | | | | |
| MR. | 10 | 30 | 1225 | 2148 | 3373 | 306.25 |
| COFFEE 60B | | | | | | |
| 8.5 Grind 30 g | | | | | | |
| AEROPRESS | 10 | 20 | 1231 | 2060 | 3291 | 307.75 |
| 60B 5.0 | | | | | | |
| Grind 14 g | | | | | | |
| CHEMEX | 5 | 14 | 1178 | 2032 | 3210 | 294.50 |
| 60B 1.0 | | | | | | |
| Grind 5 g | | | | | | |
| AEROPRESS | 7 | 14 | 1177 | 2015 | 3192 | 294.25 |
| 60B 7.0 | | | | | | |
| Grind 14 g | | | | | | |
| FRENCH | 7 | 22 | 995 | 1694 | 2689 | 248.75 |
| PRESS 60B | | | | | | |

Table 3C below shows the amount of chlorogenic acid extracted per cup of coffee and also includes total dissolved solids (TDS), which is an accurate measure of extracting a pleasant tasting cup of coffee. High TDS reflects a sour cup of coffee lacking in sweetness. Low TDS produces a bitter and dry coffee. All of the coffees in this table meet the established standard.

Brewing Method: The brewing methods in Table 3C se are the best brewing methods for flavor and extraction.

Grind Size: These range from 1-11 as settings on a Mahlkonig® grinder. 1 is very fine, 11 is very coarse. The finer, the higher the extraction Grind size was verified in a reference laboratory.

Grams of Coffee: This is the starting number of grams used for the extraction and over which water was poured.

Initial Volume: Grams of water used for the extraction from ground coffee beans. Aeropress is a smaller volume, more efficient method because of the pressure used and so requires a smaller volume.

Final Volume: This is the total amount of coffee that was extracted from the water poured over the coffee grounds. In most methods, some water was left in the coffee grounds. For the unfiltered Turkish method, however, the grounds are soaked with water so the final volume is higher.

Brew Time: This is the length of time over which the extraction occurred. Most of these times are specified by the maker of the device used.

TDS ppm: This is the best measure of how good an extraction is and is specified by the Specialty Coffee Association of America®.

Brew Temp: This is the temperature of the water we used for the extraction in brewing each cup of coffee. Turkish specifies boiling water, hence 212 F. The other methods were slightly less, 210 F.

CGAs: Three standards for CGAs were obtained for the liquid chromatography device used to assess the coffees. Amounts are shown for 3 CGA, 4 CGA and 5 CGA, which are the three key chlorogenic acids in coffee, as well as the total for 3, 4, and 5 CGA.

TABLE 3C

| Brewing | Grind | Grains of | Initial | Final | TDS ppm | TDS | Brew | 3 | 4 | 5 | 3, 4, 5 |
|---|---|---|---|---|---|---|---|---|---|---|---|
| Method | Size | Coffee | Volume | Volume | Brew Time | ppm | Temp | CGA | CGA | CGA | CGA |
| Turkish | 1 | 30 | 300 | 313.5 | 3:30 | 1910 | 212 | 545 | 298 | 223 | 1066 |
| Unfiltered | | | | | | | | | | | |
| Turkish | 1 | 30 | 300 | 258.5 | 3:30 | 1970 | 212 | 470 | 256 | 184 | 910 |
| Gold Filter | | | | | | | | | | | |
| Hario V60 | 11 | 30 | 300 | 232.5 | 7:30 | 2190 | 212 | 419 | 228 | 160 | 807 |
| Turkish | 1 | 30 | 300 | 214.0 | 3:30 | 2150 | 212 | 400 | 218 | 154 | 772 |
| Paper filter | | | | | | | | | | | |
| Able | 11 | 30 | 300 | 236.0 | 6:30 | 2140 | 210 | 392 | 210 | 151 | 753 |
| Kalita | 11 | 30 | 300 | 230.0 | 3:30 | 2070 | 210 | 357 | 193 | 138 | 688 |
| Elan | 11 | 30 | 300 | 236.5 | 3:30 | 1920 | 210 | 336 | 182 | 128 | 646 |
| Collective | | | | | | | | | | | |
| Aeropress | 3 | 22 | 200 | 178.0 | 3:30 | 2200 | 210 | 323 | 180 | 128 | 631 |
| Aeropress | 5 | 22 | 200 | 182.0 | 3:30 | 2110 | 210 | 329 | 175 | 126 | 630 |
| Aeropress | 7 | 22 | 200 | 144.5 | 3:30 | 2220 | 210 | 309 | 167 | 119 | 595 |
| Elan | 7 | 30 | 300 | 138.0 | 3:30 | 2400 | 210 | 299 | 163 | 114 | 576 |
| Collective | | | | | | | | | | | |
| Elario V60 | 11 | 30 | 300 | 186.0 | 3:30 | 2430 | 210 | 290 | 159 | 112 | 561 |
| Able | 11 | 30 | 300 | 184.5 | 3:30 | 2110 | 210 | 290 | 155 | 113 | 558 |
| Kalia | 7 | 30 | 300 | 129.0 | 3:30 | 2350 | 210 | 258 | 141 | 99 | 498 |

From the foregoing description, it can be seen that the methods of making coffee in accordance with the present invention deliver the highest amounts of phenols, chlorogenic acids, antioxidants and anti-inflammatory compounds into a cup of coffee without sacrificing flavor, as these high polyphenol coffees score among the very best tasting coffees as determined by the independent coffee review:
A. 94 Daktari Coffee, Kenya Nyeri Gathugu
Location: Burlington, Vermont
Origin: Nyeri growing region, southcentral
Kenya
Roast: Medium-Light
Est. Price: 815.00/12 ounces
Review Date: July 2016
Agtron: 54/7 5
Aroma: 9
Acidity: 9
Body: 9
Flavor: 9
Aftertaste: 8
Blind Assessment: Deep, subtle, resonant. Concord grape, fresh-cut cedar, wisteria-like flowers, black sage, lemon zest in aroma and cup. Roundly and richly bright acidity; full, syrupy mouthfeel. Flavor consolidates around grape and cedar in a continued deep, ringing finish.
Notes: Despite challenges ranging from unclear government coffee policy and urban encroachment on prime coffee lands to chronically unstable weather, the famed Kenya coffee auction system and its participating cooperatives continue to produce some of the world's most elegant and distinctive coffees. Meticulously wet-processed using traditional ferment-and-wash methods. Daktari uses sophisticated analytical chemistry to identify roast profiles aimed at retaining compounds assumed to be most health-enhancing in each coffee.
The Bottom Line: A Kenya for those who value subtle power and layered complexity. The Bottom Line: A Kenya for those who value subtle power and layered complexity.
B. 93 Daktari Coffee, Kenya Nyeri Mahiga
Location: Burlington, Vermont
Origin: Nyeri growing region, south central Kenya
Roast: Medium-light
Est. Price: S15.00/12 ounces
Review Date: July 2016
Agtron: 56/77
Aroma: 9
Acidity: 8
Body: 9
Flavor: 9
Aftertaste: 8
Blind Assessment: Crisply sweet, elegantly tart. Blackcurrant, baker's chocolate, lemon verbena, lavender in aroma and cup. Round, richly tart acidity; lightly plush mouthfeel. Blackcurrant and lemon verbena carry into a resonant finish. Notes: Despite challenges ranging from unclear government coffee policy and urban encroachment on prime coffee lands to chronically unstable weather, the famed Kenya coffee auction system and its participating cooperatives continue to produce some of the worlds most elegant and distinctive coffees. Meticulously wet-processed using traditional ferment-and-wash methods. Daktari uses sophisticated analytical chemistry to identify roast profiles aimed at retaining compounds assumed to be most health-enhancing in each coffee. The Bottom Line: Classic Kenya; sweet but seriously dry, berry-toned, elegant.
It is noted that the new and improved methods of the present invention produce high polyphenol, high chlorogenic coffee through completely natural means, i.e., nothing is added to the coffee. The results are accomplished through careful selection of the coffee beans, artisanal roasting and proper brewing, with no polyphenol degradation over time.

Exemplary embodiments have been disclosed above and illustrated in the accompanying drawings. It will be understood by those skilled in the art that various changes, omissions, and additions may be made to that which is specifically disclosed herein without departing from the spirit and scope of the present invention.

What is claimed is:

1. A method of preparing a liquid coffee drink comprising:
determining amounts of polyphenols in a plurality of coffee beans using mass spectrometry;
selecting coffee beans from the plurality of coffee beans that contain between 20,000 mg/kg and 40,000 mg/kg of 3 caffeoylquinic acid (3 CQA);
roasting the selected coffee beans at a temperature and for a time period of 7.5 to 12 minutes at a temperature of 390° F. to 412° F., wherein the roasted coffee beans have between 11,000 and 22,000 mg/kg of 3 CQA;
grinding the roasted coffee beans to produce ground coffee with a particle size of between 100 microns and 1200 microns; and
brewing the roasted around coffee with water at 210° F. for about 3.5 minutes to produce the liquid coffee drink, wherein the liquid coffee drink includes at least 258 mg of 3 chlorogenic acid per 250 mL of the liquid coffee drink, at least 141 mg of 4 chlorogenic acid per 250 mL of the liquid coffee drink, and at least 99 mg of 5 chlorogenic acid per 250 mL of the liquid coffee drink.

2. The method according to claim 1, wherein the liquid coffee drink includes at least 498 mg of 3, 4, and 5 chlorogenic acids combined.

3. The method according to claim 1, wherein the liquid coffee drink contains not more than 2400 ppm TDS.

4. A method of preparing a liquid coffee drink consisting of:
determining amounts of polyphenols in a plurality of coffee beans using mass spectrometry;
selecting coffee beans from the plurality of coffee beans that contain between 20,000 mg/kg and 40,000 mg/kg of 3 caffeoylquinic acid (3 CQA) per kg of bean;
roasting the selected coffee beans at a temperature and for a time period of 7.5 to 12 minutes at a temperature of 390° F. to 412° F., wherein the roasted coffee beans have between 11,000 and 22,000 mg/kg of 3 3 CQA;
grinding the roasted coffee beans to produce a ground coffee with a particle size of between 100 microns and 1200 microns; and
brewing the roasted ground coffee with water at 212° F. for about 3.5 minutes to produce the liquid coffee drink, wherein the liquid coffee drink includes about 500 mg of 3 chlorogenic acid per 250 mL of the liquid coffee drink, about 290 mg of 4 chlorogenic acid per 250 mL of the liquid coffee drink, and about 220 mg of 5 chlorogenic acid per 250 mL of the liquid coffee drink.

5. The method according to claim 1, wherein the liquid coffee drink includes at least 1000 mg of 3, 4, and 5 chlorogenic acids combined.

6. The method according to claim 1, wherein the liquid coffee drink contains not more than 2000 ppm TDS.
